(19) United States
(12) Patent Application Publication
(10) Pub. No.: US 2020/0282217 A1
(43) Pub. Date: Sep. 10, 2020

(54) Systems and Methods for Neuromodulation of Sympathetic and Parasympathetic Cardiac Nerves

(71) Applicant: NeuroTronik IP Holding (Jersey) Limited, St. Helier (JE)

(72) Inventors: Stephen C. Masson, Raleigh, NC (US); Michael Cuchiara, Durham, NC (US); Efrain A. Miranda, Mason, OH (US); Richard A. Glenn, Santa Rosa, CA (US); A J. Rogers, Durham, NC (US)

(21) Appl. No.: 16/840,502

(22) Filed: Apr. 6, 2020

Related U.S. Application Data
(63) Continuation of application No. 14/801,560, filed on Jul. 16, 2015.
Application No. 14/801,560 is a continuation-in-part of application No. 14/642,699, filed on Mar. 9, 2015.
(60) Provisional application No. 62/034,142, filed on Aug. 6, 2014.
(60) Provisional application No. 62/036,526, filed on Aug. 12, 2014.

(51) Int. Cl. A61N 1/36; A61N 1/05

(52) U.S. Cl. CPC A61N 1/36114; A61N 1/36014; A61N 1/36185; A61N 1/0551

(57) ABSTRACT

A catheter system configured for delivering a neuromodulation therapy, includes a first therapeutic element positionable in a first target vessel selected from the group of blood vessels consisting of the superior vena cava, left brachiocephalic vein, right brachiocephalic vein, azygos vein or azygos arch, and a second therapeutic element in a second target vessel selected from the group of blood vessels consisting of the superior vena cava, left brachiocephalic vein, right brachiocephalic vein, internal jugular vein, azygos vein or azygos arch. The system and associated method deliver therapeutic energy to at least one parasympathetic nerve fiber external to the first target vessel using the first therapeutic element, and deliver therapeutic energy to at least one sympathetic nerve fiber external to the second target vessel using the second therapeutic element.

This application is a continuation of U.S. application Ser. No. 14/801,560, filed Jul. 16, 2015, which is a continuation in part of U.S. application Ser. No. 14/642,699, filed Mar. 9, 2015, which claims the benefit of the following U.S. Provisional Applications No. 61/950,191, filed Mar. 9, 2014, No. 61/950, 208, filed Mar. 10, 2014, No. 62/034,142, filed Aug. 6, 2014, and 62/036,526, filed Aug. 12, 2014, each of which is incorporated herein by reference.

### TECHNICAL FIELD OF THE INVENTION

The present application generally relates to systems and methods for neuromodulation using elements disposed within the vasculature.

### BACKGROUND

Co-pending U.S. application Ser. No. 13/547,031 entitled System and Method for Acute Neuromodulation, filed Jul. 11, 2012 (Attorney Docket: IAC-1260; the “'031 application”), filed by an entity engaged in research with the owner of the present application, describes a system which may be used for hemodynamic control in the acute hospital care setting, by transvascularly directing therapeutic stimulus to parasympathetic nerves and/or sympathetic cardiac nerves using an electrode array positioned in the superior vena cava (SVC). In accordance with a described method, autonomic imbalance in a patient may be treated by energizing a first therapeutic element disposed in a superior vena cava of the patient to deliver therapy to a parasympathetic nerve fiber such as a vagus nerve, and energizing a second therapeutic element disposed within the superior vena cava to deliver therapy to a sympathetic cardiac nerve fiber. A disclosed neuromodulation system includes a parasympathetic therapy element adapted for positioning within a blood vessel, a sympathetic therapy element adapted for positioning with the blood vessel; and a stimulator configured to energize the parasympathetic therapy element to deliver parasympathetic therapy to a parasympathetic nerve fiber disposed external to the blood vessel and to energize the sympathetic therapy element within the blood vessel to deliver sympathetic therapy to a sympathetic nerve fiber disposed external to the blood vessel. In disclosed embodiments, delivery of the parasympathetic and sympathetic therapy decreases the patient's heart rate (through the delivery of therapy to the parasympathetic nerves) and elevates or maintains the blood pressure (through the delivery of therapy to the cardiac sympathetic nerves) of the patient in treatment of heart failure.

PCT Publication No. WO 2012/149511, entitled Neuromodulation Systems and Methods for Treating Acute Heart Failure Syndromes, and PCT Publication No. WO 2013/022532, entitled Catheter System for Acute Neuromodulation, each of which was filed by an entity engaged in research with the owner of the present application, describe therapy elements, one of which is positionable within a first blood vessel such as a superior vena cava, and the other of which is positionable in a second, different, blood vessel such as the pulmonary artery. The first therapy element is energized to deliver neuromodulation therapy to a parasympathetic nerve fiber such as a vagus nerve, while the second therapy element is energized to deliver neuromodulation therapy to a sympathetic nerve fiber such as a sympathetic cardiac nerve fiber. For treatment of acute heart failure syndromes, the neuromodulation therapy may be used to lower heart rate and increase cardiac inotropy.

The present application describes catheter systems and methods suitable for carrying out therapy of the type disclosed in the above-referenced applications.

### BRIEF DESCRIPTION OF THE DRAWINGS

FIG. 1A is an anatomical drawing schematically illustrating exemplary positions for placement of therapeutic elements so as to capture target sympathetic and parasympathetic nerves in accordance with methods disclosed herein.

FIG. 1B schematically illustrates exemplary positioning of a catheter system to place separate therapeutic elements in separate vessels.

FIG. 2A is a perspective view of an embodiment of a catheter system suitable for positioning as shown in FIG. 1B.

FIG. 2B is a cross-section view taken along the plane designated 2B-2B in FIG. 2A.

FIG. 2C is a side elevation view showing the catheter system of FIG. 2A within an introducer sheath. The introducer sheath is shown in cross-section to allow the catheter system to be easily seen.

FIG. 3 illustrates a second embodiment of a catheter system positionable to place separate therapeutic elements in separate vessels. The system is schematically shown with therapeutic elements positioned in the left brachiocephalic vein and superior vena cava.

FIG. 4 illustrates an embodiment of a catheter system positionable to place separate therapeutic elements in a common vessel. The system is schematically shown with therapeutic elements positioned in the left brachiocephalic vein.

FIG. 5A illustrates an embodiment of a catheter system positionable to place a portions of single therapeutic element support in two separate vessels. The system is schematically shown with a portion of the therapeutic element support positioned in the left brachiocephalic vein and a portion positioned in the right brachiocephalic vein.

FIG. 5B is similar to FIG. 5A, but shows the system with a portion of the therapeutic element support positioned in the left brachiocephalic vein and a portion positioned in the superior vena cava.

FIGS. 6A and 6B illustrate use of an anode in one vessel and a cathode in a second vessel to create an electric field that captures target nerves within the brachiocephalic triangle.

FIG. 7 is a perspective view of an exemplary electrode carrying member; FIG. 8A is a side elevation view of a strut of the electrode carrying member of FIG. 7;

FIG. 8B is a cross-section view of the strut of FIG. 8A, taken along the plane designated A-A in FIG. 8A;

FIG. 8C is an alternative to the strut cross-section of FIG. 8B;

FIG. 8D is another alternative to the strut cross-section of FIG. 8B;

FIG. 9A is a distal end view of the therapeutic element of FIG. 7;

FIG. 9B is similar to the distal end view of FIG. 9A but shows an alternative strut arrangement;

FIG. 10 is a perspective view of an alternative electrode carrying member;

FIGS. 11A is a side elevation view of the electrode carrying member of FIG. 10

FIGS. 11B and 11C are similar to FIG. 11A but show the electrode carrying member in a blood vessel. FIG. 11C illustrates the electrode carrying member with the inner member in the withdrawn position.

FIG. 12 is a perspective view of a third embodiment of an electrode carrying member; FIG. 13 is a perspective view of a fourth embodiment of an electrode carrying member.

### DESCRIPTION

The present application describes catheter systems and methods which may be used for acute heart failure syndrome (“AHFS”) treatment or for other therapeutic purposes. The systems and methods disclosed herein can be used to deliver therapy to decrease or sustain the patient's heart rate (such as through the delivery of therapy to the parasympathetic nerves) and elevate or maintain the patient's blood pressure (through the delivery of therapy to the cardiac sympathetic nerves) of the patient in treatment of heart failure, as well as for other therapeutic effects. The therapy can result in increased cardiac inotropy and improved cardiac output while lowering or maintaining the heart rate. In the disclosed methods, the therapy is delivered from therapeutic elements positioned in blood vessels at locations that are superior to the heart.

The catheter system includes first therapeutic elements for parasympathetic nerve fiber (e.g. vagus nerve fiber) neuromodulation, and second therapeutic elements for cardiac sympathetic nerve fiber neuromodulation. The first and second therapeutic elements may be positioned in the same blood vessel or in separate blood vessels. A neuromodulation system employing the disclosed types of catheter systems includes an external pulse generator/stimulator (not shown) that is positioned outside the patient's body (although in modified embodiments an implantable stimulator may instead be used, in which case the percutaneous catheter systems disclosed herein may be replaced with leads). The stimulator/pulse generator is configured to energize the first therapeutic element to deliver parasympathetic therapy to an extravascular parasympathetic nerve fiber, and to energize the second therapeutic element to deliver sympathetic therapy to an extravascular sympathetic nerve fiber. The first and second therapeutic elements are carried by percutaneous catheters that are coupled to the external pulse generator.

The present inventors have identified vascular locations from which beneficial neuromodulation or stimulation can be transvascularly delivered to target nerves so as to carry out the therapy described herein. As discussed above, this therapy may lower or sustain the heart rate while elevating or maintaining the blood pressure, and can result in increased inotropy and improved cardiac output.

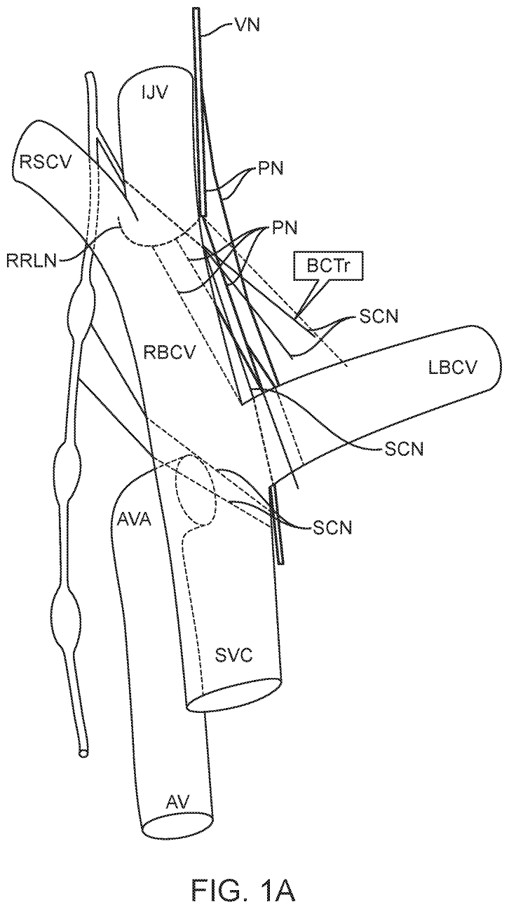

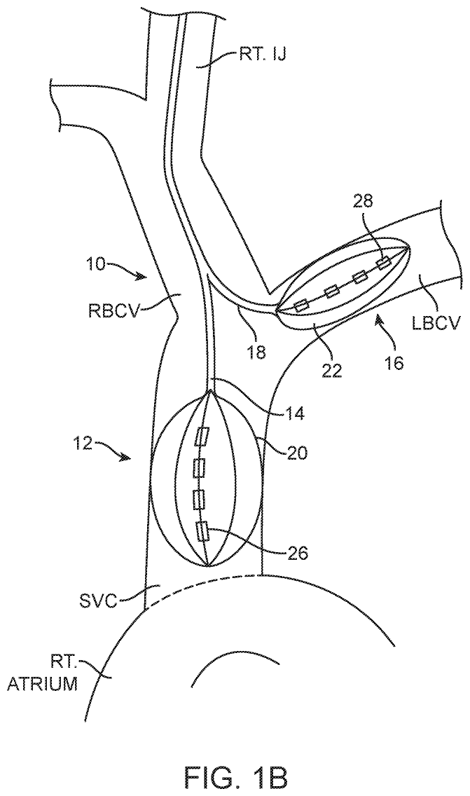

FIG. 1A illustrates the venous anatomy in the region of interest and shows the locations of parasympathetic nerves PN (lighter/yellow colored lines) and sympathetic (darker/purple colored lines) cardiac nerves SCN within the region of interest. Dashed dark/purple and light/yellow colored lines indicate such nerves passing behind vessels. The drawing is additionally labeled as follows:

| | |
|---|---|
| AV | Azygos Vein |
| AVA | Arch of Azygos Vein |
| BCTr | Brachiocephalic Triangle |
| IJV | Internal Jugular Vein |
| LBCV | Left Brachiocephalic Vein |
| RBCV | Right Brachiocephalic Vein |
| RRLN | Right Recurrent Laryngeal Nerve |
| RSCV | Right Subclavian Vein |
| SVC | Superior Vena Cava |
| VN | Vagus Nerve |

The vagus nerve (VN) is found in the carotid sheath in a groove between the internal jugular vein (IJV) and the common carotid artery (not depicted). As it passes anterior to the origin of the subclavian artery, it gives off the right recurrent laryngeal nerve (RRLN) forming a loop. In a fluoroscopic image, this loop would be just posteromedial to the origin on the right brachiocephalic vein (RBCV). It is a useful reference for identifying the apex of the brachiocephalic triangle (BCTr).

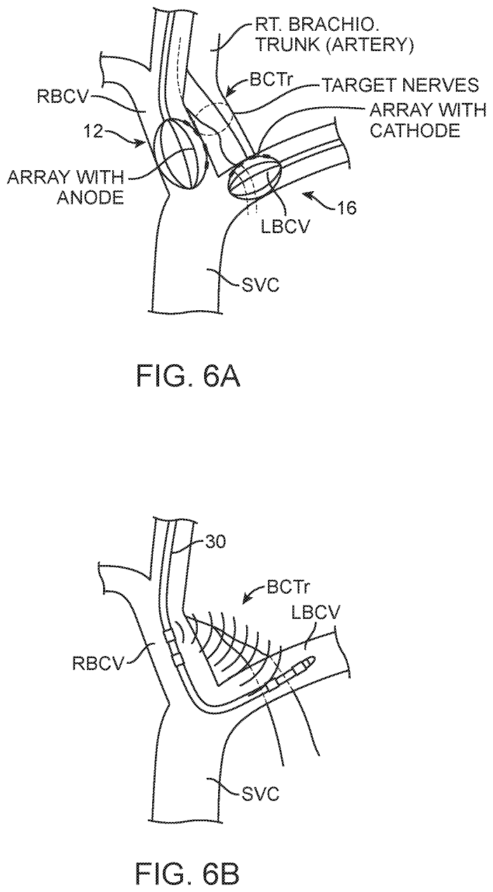

The brachiocephalic triangle (BCTr) has been identified by the present inventors as a roughly triangular region having as an inferior boundary the LBCV, a medial boundary formed by the lateral aspect of the brachiocephalic trunk (not shown but see the dashed black line and also see FIG. 6A), and a lateral wall formed by the medial aspect of the RBCV. It has an anterior wall formed by the fatty mass of the thymus gland remnants.

The apex of the BCTr lies at the origin of the right subclavian artery (RSCA) as shown. The posterior wall of the BCTr is complex and formed partly by the arch of the aorta in its inferomedial aspect, and the trachea and bronchial bifurcation in its middle region. Towards the apex of the BCTr, the posterior wall deepens with no clear boundary, formed by connective tissue, and fatty tissue containing lymphatic vessels and lymphatic nodes related to the right-sided lymphatic drainage of the head, neck, and right upper extremity. It is within this fatty mass that most of the cardiac sympathetic nerves and cardiac branches of the vagus nerve traverse the BCTr.

Based on the present inventors' findings, locations of parasympathetic nerve fibers and cardiac sympathetic nerves that can be modulated from the nearby venous vasculature to achieve the desired therapy include (1) the region of the apex of the BCTr, which region includes (as shown in FIG. 1A), lower regions of the internal jugular vein (IJV) and upper regions of the right brachiocephalic vein (RBCV); (2) in an area found in proximity to (e.g. within 1-2 cm of) the distal end of the left brachiocephalic vein (LBCV); and (3) at the superior portion of SVC (e.g. near the confluence of the right brachiocephalic vein (RBCV) and the LBCV).

Without limiting the scope of the claims, the present inventors have found that intravascular electrode positions that may be used to capture the nerves identified within regions (1)-(3) include:
positions within the RBCV, such as on the postero-medial side in proximity to the apex of the BCTr, for targeting either or both parasympathetic nerve fibers (such as, for example, the thoracic cardiac branch of the vagus nerve or nearby branches of the vagus nerve) and sympathetic cardiac nerve fibers;
other positions in proximity to the apex of the BCTr, such as the lower region of the IJV. As two non-limiting examples, the lower 1 cm or the lower 2 cm of the IVJ might be suitable electrode locations;
positions within the LBCV, such as within the first 2 cm of the LBCV from the bifurcation at the SVC or RBCV (referred to herein as the “distal” part of the LBCV), for targeting either or both parasympathetic nerve fibers and sympathetic cardiac nerve fibers. In one specific example, sympathetic cardiac nerve capture may be achieved from the posterior side, and parasympathetic nerve capture may be achieved from anterior and/or posterior positions;
positions within the SVC, particularly in the superior portion, from which either or both types of nerves can be captured using posterior or postero-medial electrodes. In one specific example, sympathetic cardiac nerves may be captured using posteriorly positioned intravascular electrodes while vagal branches (parasympathetic) can be captured using postero-medially positioned intravascular electrodes;
in the azygos vein (AV) or arch of the azygos vein (AVA), for targeting either or both parasympathetic nerve fibers or sympathetic cardiac nerve fibers.

Therapy targeting only sympathetic cardiac nerve fibers or parasympathetic cardiac nerve fibers can also be achieved from the identified regions. For example, sympathetic cardiac nerve capture from the identified sites might be used without accompanying parasympathetic capture, in order to elevate or sustain blood pressure and/or to increase inotropy.

Nerve fibers that may be captured from venous locations superior to the heart (including the locations listed above) include, without limitation, parasympathetic and/or sympathetic nerve fibers that are coursing towards the cardiac plexus and/or that innervate the heart via the cardiac plexus, sympathetic nerve structures including the right dorsal medial cardiopulmonary nerve, the right dorsal lateral cardiopulmonary nerve and the right stellate cardiopulmonary nerve, and vagal nerve structures including the right cranial vagal cardiopulmonary nerve and right caudal vagal cardiopulmonary nerve. Capturing these nerves using therapeutic elements positioned in the upper venous vasculature, rather than at sites closer to the heart, allows the desired therapy to be performed from vascular locations that are safe and readily accessible.

While this application focuses on the use of intravascular electrodes for transvascular neuromodulation, it should be appreciated that electrodes may be placed directly into contact with the target nerves in the identified regions (using cuffs or other means) so as to achieve the therapy using direct rather than transvascular neuromodulation.

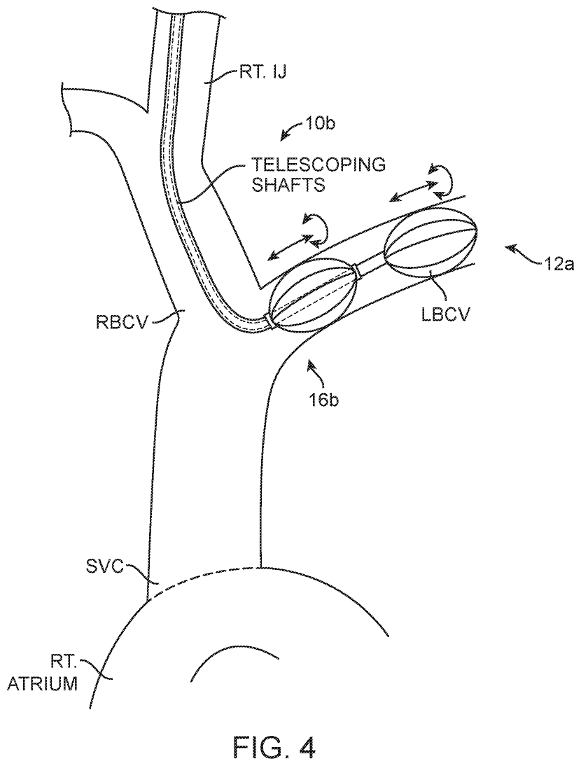

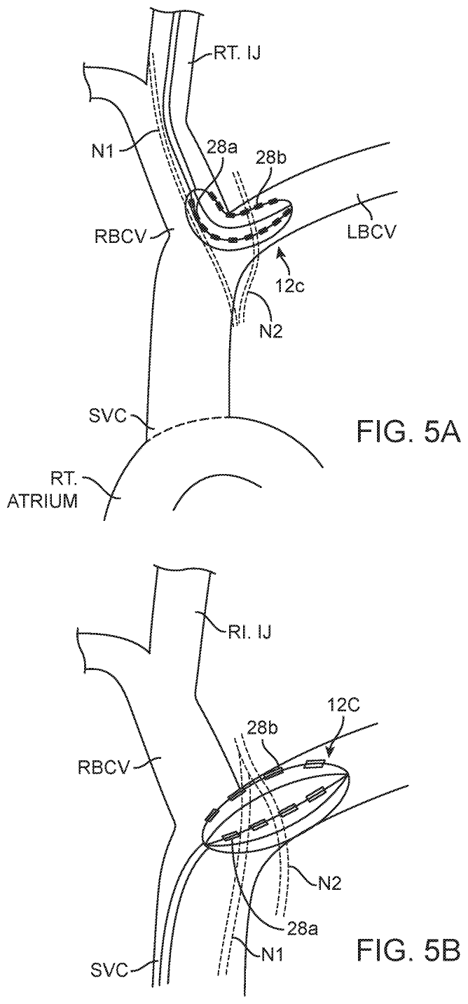

Using the identified sites, a method of delivering a neuromodulation therapy may include positioning a first therapeutic element in a first target vessel selected from the group of blood vessels consisting of the superior vena cava, left brachiocephalic vein, right brachiocephalic vein, internal jugular vein, azygos vein or azygos arch and positioning a second therapeutic element in a second target vessel selected from the group of blood vessels consisting of the superior vena cava, left brachiocephalic vein, right brachiocephalic vein, internal jugular vein, azygos vein or azygos arch. Therapeutic energy is delivered to at least one parasympathetic nerve fiber external to the first target vessel using the first therapeutic element; and therapeutic energy is delivered to at least one sympathetic nerve fiber external to the second target vessel using the second therapeutic element. In some embodiments, the first and second therapeutic elements are in different vessels (see, e.g. FIG. 1B), while in other embodiments the first and second therapeutic elements are in a common vessel (see e.g. FIG. 4). The first and second therapeutic elements may be on separate supports or electrode carrying members as in FIGS. 1B, 3 and 4, or on a common support or electrode carrying member as in FIGS. 5A and 5B.

Because the present inventors have identified the left brachiocephalic vein LBCV as a site from which sympathetic and/or parasympathetic neuromodulation may be delivered to achieve the effects disclosed in the '031 application and herein, catheter system embodiments shown in the drawings of the present application will be described in the context of use of the system to deliver at least the sympathetic stimulus, and optionally also the parasympathetic stimulus, using therapeutic elements with the LBCV. However, the disclosed catheter systems may be positioned in any combination of the vessels listed herein, or in alternate vessels or combinations of vessels to deliver stimulus to target nerve fibers outside those vessels.

FIG. 1B schematically illustrates a portion of a heart and superior vasculature, in which a right atrium RA, superior vena cava SVC, right brachiocephalic vein RBCV, left brachiocephalic vein LBCV, and right internal jugular vein RtIJ are shown. In the illustrated catheter system 10, one or more first therapeutic elements 12 are mounted to a first catheter member 14 for parasympathetic fiber (e.g. vagus nerve) neuromodulation, and one or more second therapeutic elements 16 are mounted to second catheter member 18 for sympathetic fiber neuromodulation.

The first therapeutic elements 12 (also referred to herein as the parasympathetic therapeutic elements) are energizable to modulate parasympathetic nerve fibers located outside the vasculature by directing energy to parasympathetic nerve fibers from within the SVC. The second therapeutic elements 16 (referred to as the sympathetic therapeutic elements) are energizable to modulate sympathetic nerve fibers by directing energy to sympathetic nerve fibers from within the LBCV.

In preferred embodiments, the first and second therapeutic elements 12, 16 are electrodes or electrode arrays, although it is contemplated that other forms of therapeutic elements (including, but not limited to, ultrasound, thermal, or optical elements) may instead be used. The therapeutic elements are positioned on flexible catheters.

The catheters include features expandable within the vasculature for biasing the electrodes into contact with the interior surfaces of the blood vessels so as to optimize conduction of neuromodulation energy from the electrodes to the target nerve fibers outside the vessel. The expandable features also serve to anchor the catheter and electrodes at the desired position for the duration of the treatment. In the embodiments shown, the first and second therapeutic elements 12, 16 are electrode arrays carried on respective therapeutic element supports (also referred to as electrode carrying members) 20, 22 positioned on the catheter members 14, 18. Each electrode carrying member has a compressed, streamlined position for pre-deployment passage of the catheter and electrode carrying member through the vasculature during advancement of the therapeutic elements towards the target deployment site. Each electrode carrying member is expandable to an expanded position in which at least a portion of the electrode carrying member is radially deployed towards the interior wall of the blood vessel so as to bias the electrode(s) into contact with the vessel wall. A compressive sheath of the type known in the art may be positioned over the electrode carrying member to maintain the compressed streamline position, and then withdrawn to allow it to expand.

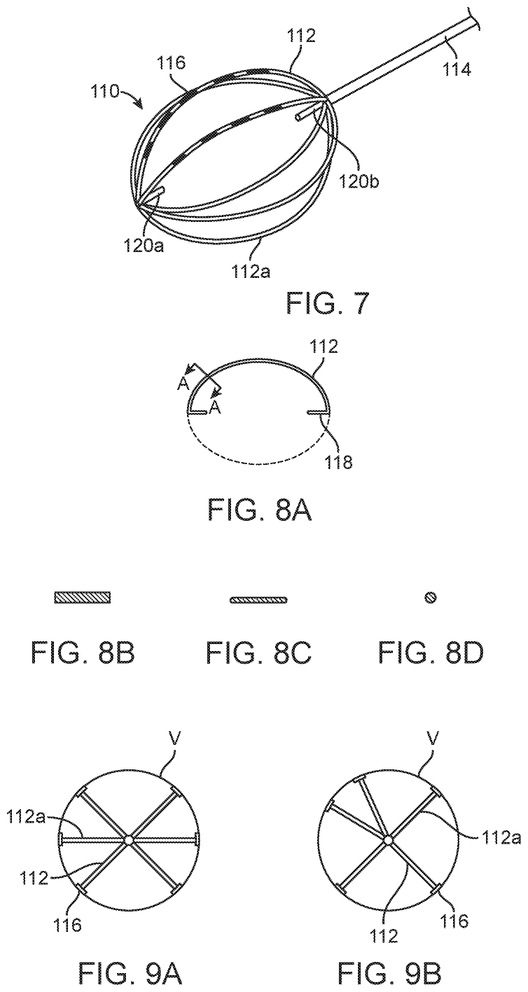

The drawings show electrode carrying members 20, 22 constructed of struts or spline elements 24 formed of resilient material such as nitinol, stainless steel, elgiloy, MP35N alloy, resilient polymer or another resilient material. The spine elements are moveable to deployed positions in a manner known in the art, to cause the spine elements to bow or extend outwardly when the electrode carrying member is moved to the expanded position. Expansion methods that may be used for this purpose include self-expansion due to shape setting of the materials, as well as using active deployment features included on the catheter. Electrodes 26, 28 are positioned on the spline elements. The electrodes can be the splines themselves, or conductive regions of the splines where the remaining portions of the splines are covered or coated with insulative material. Alternatively, electrodes may be attached to the splines, or printed or plated onto the splines. The number and the arrangement of splines are selected to optimize positioning of the electrodes within the target blood vessel. Additional features that may be found on the electrode carrying members are found in the description of FIGS. 7 through 13.

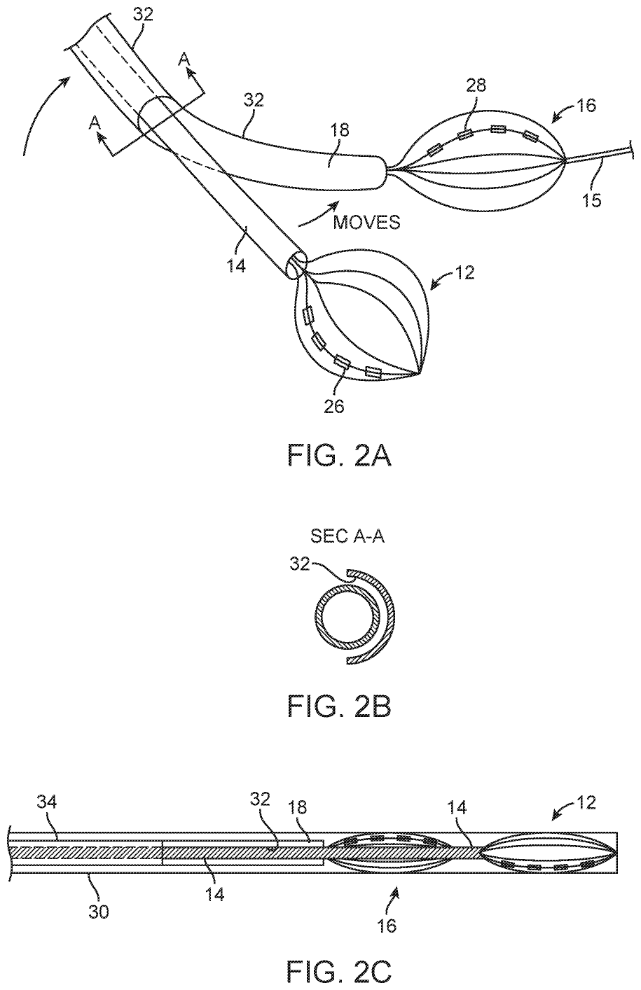

The catheter system is designed such that catheter members 14, 18 and their associated therapeutic elements are percutaneously introduced (e.g. using access through the femoral vein, subclavian, or internal jugular vein). FIGS. 2A through 5 show embodiments of telescoping catheter systems, in which one of the catheter members telescopes over or through the other of the catheter members for ease of use.

FIG. 2A shows a first embodiment of a catheter system 10 extending from an introducer sheath 30. In the system 10, a distal portion of the catheter member 18 has a recess or concave surface 32, allowing the distal portion of the catheter member 14 to nest within the recess so that the two catheter members 14, 18 are generally coaxially aligned as shown in the cross-section of FIG. 2B. In this example, the recess/concave surface is created by forming the catheter member 18 to have a generally C-shaped cross-section, which may be an arc of a circle. The recess may extend the full length of the catheter, or it may be only at the distal section, with the proximal section 34 being tubular with a lumen that receives the proximal section of the catheter member 14 in telescoping fashion as shown in FIG. 2C. The catheter members can thus be compactly arranged and positioned together within the introducer sheath 30 as shown in FIG. 2C. When the system is positioned in the region where the LBCV and SVC bifurcate, the sheath can be withdrawn to allow the catheter member 18 to separate from the catheter member 14 so that the therapeutic element 16 can be advanced into the LBCV (over a guidewire 15 if needed) and the element 12 into the SVC. The telescoping relationship of the catheter members 14, 18 allows the longitudinal position of each therapeutic element within its corresponding vessel to be independently adjusted during mapping or therapy as needed for optimal nerve capture.

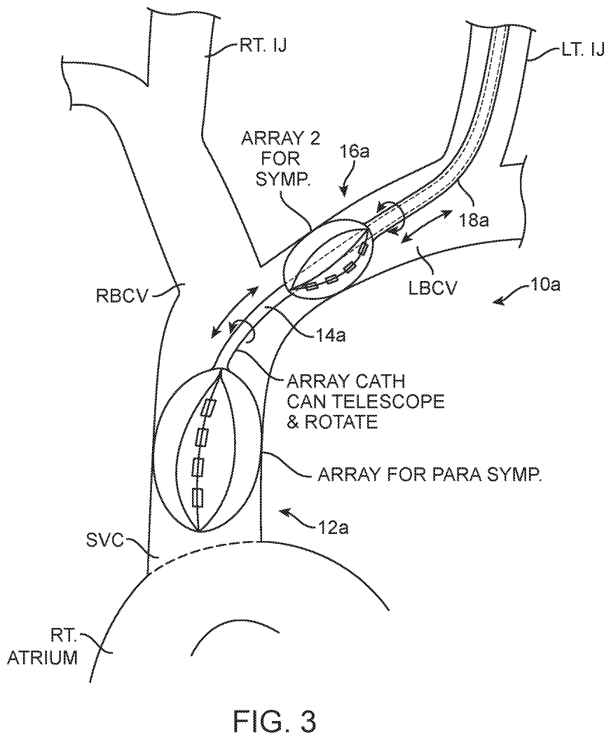

A second embodiment of a catheter system 10a is shown in FIG. 3 and also may be used to position separate therapeutic elements in each of the SVC and LBCV. This configuration allows introduction of the catheter system 10a into the vasculature via the left internal jugular vein (LTIJ) or another vein leading into the LBCV. As with the catheter system of the first embodiment, the catheter system 10a includes telescoping catheter members 14a, 18a, each having a therapeutic element 12a, 16a. The catheter members 14a, 18a share a common longitudinal axis, such that the catheter member 14a runs through the therapeutic element 12a and extends from its distal end. The catheter members 14a, 18a may be independently translated (longitudinally) and rotated (relative to the longitudinal axis), allowing for independent longitudinal and rotational positioning of the therapeutic elements for optimal delivery of therapy.

The embodiment of FIG. 3 may be adapted for use with a femoral approach into the vasculature, optionally using a guidewire. In such a variation, the distal-proximal positioning of the therapeutic elements 12a, 16a is reversed, with the therapeutic element 16a to be positioned in the LBCV positioned distally to the therapeutic element 12a to be positioned in the SVC.

The embodiment of FIG. 3 may also be used to position a therapeutic element in the RBCV and another therapeutic element in the SVC.

Referring to FIG. 4, a catheter system 10b similar to that shown in FIG. 3 may be employed to capture two different nerve targets from within a single vessel. For example, therapeutic elements 12b, 16b may both be positioned within the LBCV as shown or in the SVC (not shown), or in the RBCV, or in the AV or AVA, (also not shown) with one positioned to capture a parasympathetic nerve and the other positioned to capture a cardiac sympathetic nerve. As discussed, the design of the catheter system allows the therapeutic elements 12b, 16b to be independently positioned both longitudinally and radially.

In yet another alternative embodiment shown in FIG. 5A, a single therapeutic element support 12c is positioned across multiple vessels so as to capture multiple nerves (e.g. different nerves from different vessels). For example, FIG. 5A shows therapeutic element 12c positioned such that a first portion having first electrodes 28a is disposed within the RBCV and captures first nerve N1 (which may be, for example, a parasympathetic nerve), and a second portion having second electrodes 28b is disposed within the LBCV and captures second nerve N2 (e.g. a cardiac sympathetic nerve). In a modified position, either the first portion or the second portion might instead be within the superior portion of the SVC, with first electrodes 28a capturing nerve N1 from within the SVC and second electrodes 28b capturing nerve N2 from within the LBCV.

See FIG. 5B. Note that in the FIG. 5B embodiment, the therapeutic element support 12c may be positioned such that when it is deployed it can sit in its natural elongated deployed shape.

The embodiments described above may also be used to deliver therapy where one or both of the therapeutic elements is within the azygos system (which includes the azygos vein AV and the azygos arch AVA). For example, using modifications of the above embodiments or using therapeutic elements on separate catheters, a therapeutic element might be disposed in the AV or AVA for delivering therapy to cardiac sympathetic nerves, and another therapeutic element (or, if a therapeutic element support of the type shown in FIG. 5B is positioned in the AV or AVA, a part of that support) might be disposed in the AVA, SVC, LBCV, or RBCV for use in delivering therapy to parasympathetic nerve fibers. As another example, capture of parasympathetic nerve fibers and sympathetic cardiac nerve fibers for achieving the therapy disclosed above can be achieved using a single therapeutic element in the AVA, or a pair of therapeutic elements in the AVA, where one such element is positioned to capture the parasympathetic nerve targets and the other is positioned to capture the cardiac sympathetic nerve targets.

Catheter systems may also be used to direct an electric field from one vessel to another to capture nerve targets in tissues disposed along the path of the electric field. Such an arrangement is particularly useful for capturing nerve targets located within the BCTr. To capture nerves in the BCTr, one or more electrodes positioned in one of the vessels are used as the anode and one or more electrodes positioned in the other vessel are used as the cathode. In FIG. 6A, electrodes 16 positioned in the LBCV function as the cathode while electrodes 12 positioned in the RBCV function as the anode, although the polarities can be reversed such that the electrodes in the LBCV function as the anode. The electrode field resulting from activation of the electrodes passes through the BCTr and can be used to capture nerves within the BCTr.

While the FIG. 6A embodiment uses a pair of therapeutic elements separately positioned within the two vessels, another useful configuration comprises a catheter 30 equipped with multiple electrodes or electrode sets as shown in FIG. 6B, where the catheter is positionable to place one electrode/electrode set in one vessel and the other electrode/electrode set in the other vessel (e.g. using a guidewire or steerable features of the catheter). The catheter system of this embodiment might additionally include features such as anchors expandable into contact with one or both of the vessel walls to maintain the catheter position once it has been placed at the desired location, and then later retractable to permit removal of the catheter from the vasculature. Alternate designs that can be used in place of the FIG. 6B design include the telescoping catheter systems of FIGS. 3 and 4 or the system of FIG. 5A, each of which would be operated with one therapeutic element serving as the cathode in one vessel and the other serving as the anode in the other vessel to direct an electric field through the BCTr.

Anode/cathode devices such as those shown in FIGS. 6A and 6B might be used in other pairs of vessels to generate an electric field that captures nerve targets in tissues disposed along the path of the electric field. Other combinations of vessels that might be used in a similar fashion, where either one of the listed sites is used as the anode location, and the other is used as the cathode location, include: SVC and RBCV, SVC and AV, SVC and AVA, RBCV and AV, or RBCV and AVA.

In a further modification to the FIG. 6A and FIG. 6B embodiments, an anode might be positioned in a first vessel, a first cathode positioned in a second vessel, and second cathode positioned in a third vessel. In use, a parasympathetic nerve fiber may be captured by the electric field created between the first and second vessel, and a sympathetic nerve fiber may be captured by the electric field created between the first and third vessel. For example, the anode might be positioned in the SVC, with the first cathode in the LBCV and the second cathode in the RBCV.

The catheter systems are provided with instructions for use instructing the user to position and use the systems in delivering therapy to a patient in accordance with the methods described herein.

FIGS. 7 through 13 show electrode carrying members (also referred to here as “devices”) that may be used in any of the described embodiments, or in alternative systems in which individual electrode carrying members on separate catheters are used for each target blood vessel. The device 110 includes a plurality of spaced-apart longitudinally-extending struts 112, 112a positioned on the end of a catheter shaft 114. The struts 112, 112a are pre-shaped to give the device 110a predetermined shape. One or more of the struts carries one or a plurality of electrodes 116 on its outward-facing surface, which is the surface that will contact the interior wall of the vessel when the electrode carrying member is expanded within the vessel. Other struts, also referred to as support struts 112a, are free of electrodes that will deliver stimulus.

A side elevation view of one strut 112 is shown in FIG. 8A. As shown, the strut is shape set to an arcuate shape. Opposite ends of the strut include inwardly-extending distal and proximal members 118. In the assembled electrode carrying member, the distal ones of the members 118 are bundled or attached together, and the proximal ones of the members 118 are bundled or attached together, forming distal and proximal hubs 120a, 120b (FIG. 7). Positioning the hubs within the three-dimensional geometry defined by the struts 112, 112a helps minimize the length of the device. It also provides a pivot point for the device within its own framework so the device can contour to the shape of the vessel despite its connection to a catheter shaft 114.

FIGS. 9A and 9B are distal end views of the device disposed in a vessel whose wall is labeled V. The collection of the struts 112, 112a may have uniform spacing around the circumference of the device as in FIG. 9A, or non-uniform spacing as in FIG. 9B, depending on the relative locations of the target nerves to be captured using the electrodes on the device.

The cross-sectional shape of the struts 112, 112a in the lateral direction may be generally rectangular as shown in FIG. 8A, or some alternative elongated shape that includes a long edge that is outward-facing and generally flat. This geometry provides a generally flat surface for attachment of electrodes, while allowing the strut to be sufficiently thin to minimize its cross-sectional area witin the blood vessel. The rectangular or elongated shape additionally provides flexiblity in the radial direction while providing lateral stability in the circumferential direction. Alternative shapes may be used to provide better hemodynamic response by rounding the edges of the rectangular shape (FIG. 8B) or by giving the cross-section a round (FIG. 8C) or more rounded cross-section.

The device 110 is designed to bias the electrodes into contact with the vessel wall. The pre-shaped electrode carrying member 110 is set so that its natural expanded shape (the shape it would assume if expanded outside of the patient) has a diameter that is larger than the diameter of the vessel for which it is intended. Thus when the electrode carrying member is expanded in the intended vessel, it will assume a shape that differs from its natural expanded shape, and its expansion forces will push the electrodes against the vessel walls.

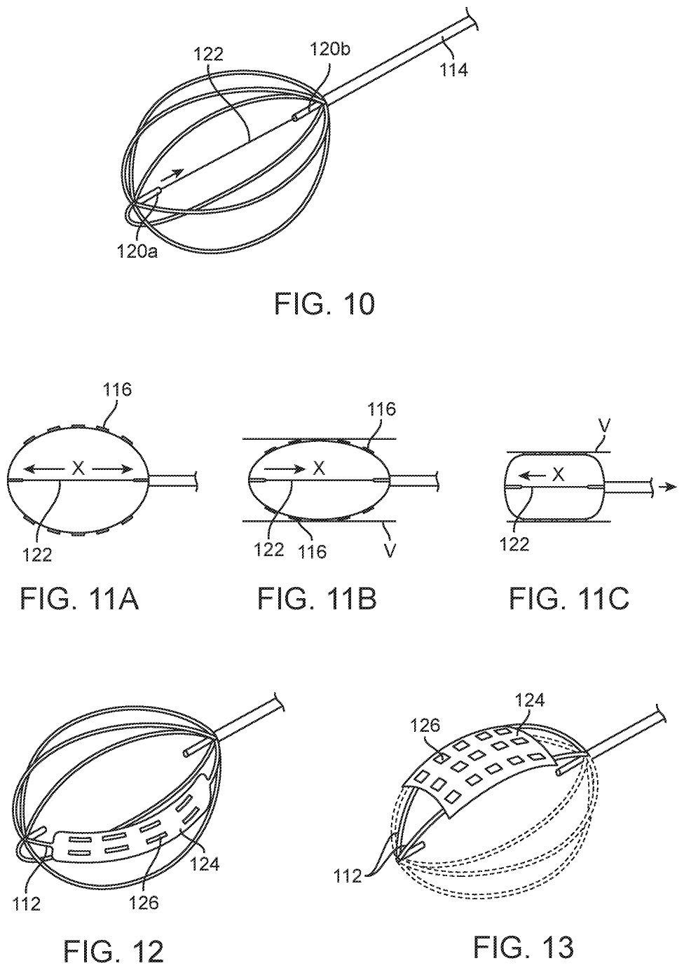

An inner member 122 may extend proximally from distal hub 120 into catheter as shown in FIG. 10. Inner member 122 may be flexible or more rigid. As schematically shown in FIG. 11A, when expanded in an unconstrained environment, the longitudinal length of the electrode carrying member 110 is X. However, when the electrode carrying member is expanded in a blood vessel, the wall V of the blood vessel constrains its radial expansion, leaving it in a more elongated shape with a longitudinal length that is greater than X. This can prevent some of the electrodes on the struts from contacting the vessel wall V, as shown in FIG. 11B. To bring those electrodes into contact with the vessel wall, the inner member 122 can be withdrawn in a proximal direction as indicated by the arrow in FIGS. 10 and 11C, drawing the distal hub closer to the proximal hub. This shortens the longitudinal length of the device so that it is equal or less than X, and in doing so increases the diameter of the device, pressing a larger number of electrodes into contact with the vessel wall as shown in FIG. 11C.

The electrodes 116 may be carried by the struts 112 in a variety of ways. For example, the electrodes may be mounted to or formed onto a substrate that is itself mounted onto a strut or a plurality of struts, or the struts might be flex circuits including the electrodes, or the electrodes might be formed or deposited directly onto the struts. As discussed, the material forming the struts 112 may have a shape set or shape memory that aids in biasing the circumferentially-outward facing surfaces (and thus the electrodes) against the vessel wall. The struts 112 or substrates might utilize materials or coatings that allow the electrodes' active surfaces (those intended to be placed against the vascular wall) to be exposed, but that insulate the remainder of each electrode's surface(s) against loss of stimulation energy into the blood pool. In some embodiments, the struts 112 or substrate may be formed of an insulative substrate such as a polymer (including silicone, polyurethanes, polyimide, and copolymers) or a plastic. The electrodes can be constructed onto the strut or substrate using a variety of manufacturing techniques, including subtractive manufacturing processes (such as mechanical removal by machining or laser cutting), additive processes (such as laser sintering, deposition processes, conductor overmolding), or combinations (such as printed circuit technology with additive plating). In some embodiments, the struts and electrodes may be flex circuit or printed circuit elements.

As shown in FIG. 12, a substrate 124 having multiple rows of electrodes 116 may be placed on one strut 112 having a smaller lateral dimension than the substrate 124. Different electrode densities and patterns may be beneficial based on the type and location of the nerve fibers that are to be targeted, and multi-electrode arrays of this type allow electrode pairs to be chosen based on the desired direction of the current needed to capture the target nerve fibers. As shown in FIG. 13, struts 112 may be placed close together to support a relatively large substrate, such as the one having multiple rows and columns of electrodes shown in the drawing.

All patents and patent applications referred to herein, including for purposes of priority, are incorporated herein by references for all purposes.

We claim:

1. A method of treating a patient, comprising:
delivering a neuromodulation therapy to increase cardiac inotropy while maintaining or lowering heart rate, said therapy including
(a) positioning a first therapeutic element in a first target vessel selected from the group of blood vessels consisting of the left brachiocephalic vein and the right brachiocephalic vein;
(b) delivering therapeutic energy to at least one parasympathetic nerve fiber external to the first target vessel using the first therapeutic element; and
(c) positioning a second therapeutic element in a second target vessel selected from the group of blood vessels consisting of the left brachiocephalic vein and the right brachiocephalic vein; and
(d) delivering therapeutic energy to at least one cardiac sympathetic nerve fiber external to the second target vessel using the second therapeutic element.

2. The method of claim 1, further including introducing a catheter system into the vasculature, the catheter system having the first and second therapeutic elements thereon, and advancing the catheter system within the vasculature to position the first and second therapeutic elements within the first and second target vessels, respectively.

3. The method of claim 1, wherein steps (b) and (d) are performed simultaneously.

4. The method of claim 1 wherein steps (b) and (d) are performed at separate times.

5. The method of claim 1, wherein the first and second therapeutic element comprise electrodes, and wherein steps (b) and (d) include energizing the corresponding electrodes.

6. The method of claim 1, where the first and second target vessels are both the left brachiocephalic vein or the right brachiocephalic vein.

7. The method of claim 1 wherein one of the first and second blood vessels is the right brachiocephalic vein and the other of the first and second blood vessels is the left brachiocephalic vein.

8. The method of claim 1, wherein the first therapeutic element and the second therapeutic element are separate electrodes or electrode arrays on a common electrode carrying member.

9. The method of claim 1, wherein the first therapeutic element and the second therapeutic element are separate electrodes or electrode arrays on separate electrode carrying members.

10. The method of claim 11, wherein one electrode carrying member includes a catheter member telescopingly slidable relative to a catheter member of the other electrode carrying member.

11. The method of claim 1, wherein step of delivering a stimulation therapy includes delivering a stimulation therapy to sustain or increase the blood pressure while decreasing or maintaining the heart rate.

12. A neuromodulation system comprising:
a catheter system comprising:
at least one catheter member configured for percutaneous placement into human vasculature, and at least one therapeutic element support on the at least one catheter member;
a first therapeutic element on the at least one therapeutic element support and positionable in a first target vessel selected from the group of blood vessels consisting of the left brachiocephalic vein or the right brachiocephalic vein, and
a second therapeutic element on the at least one therapeutic element support and positionable in a second target vessel selected from the group of blood vessels consisting of the left brachiocephalic vein or the right brachiocephalic vein;
wherein the at least one therapeutic element support is expandable to an expanded position to place the first therapeutic element in contact with an interior wall of the first target vessel and to place the second therapeutic element in contact with an interior wall of the second target vessel; and
a stimulator configured to deliver neuromodulation therapy to increase inotropy while lowering or maintaining heart rate and while elevating or sustaining blood pressure, wherein
the stimulator is configured to, when the at least one therapeutic element support is in the expanded position, energize the first therapeutic element within the first target vessel to deliver sympathetic therapy to a cardiac sympathetic nerve fiber disposed external to the first target vessel, to elevate or maintain the blood pressure of the patient, and
the stimulator is configured to, when the at least one therapeutic element support is in the expanded position, energize the second therapeutic element within the second target vessel to deliver parasympathetic therapy to a parasympathetic nerve fiber disposed external to the second target vessel, to lower or maintain the heart rate of the patient
wherein the first therapeutic element is arranged on the at least one therapeutic element support to deliver the sympathetic therapy from a posterior position of the first target vessel, and the second therapeutic element is arranged on the at least one therapeutic element support to deliver the parasympathetic therapy from a posterior portion of the second target vessel.

13. The method of claim 12, wherein the first therapeutic element and the second therapeutic element are separate electrodes or electrode arrays on a common therapeutic element support.

14. The method of claim 12, wherein the first therapeutic element and the second therapeutic element are separate electrodes or electrode arrays on separate therapeutic element supports.

15. The method of claim 14, wherein one electrode carrying member includes a catheter member telescopingly slidable relative to a catheter member of the other electrode carrying member.

16. The system of claim 12, wherein
one of the first and second therapeutic elements is a cathode and the other of the first and second therapeutic elements is an anode; and
the stimulator is configured to generate an electric field using the cathode and anode to modulate the sympathetic and parasympathetic cardiac nerve fibers.

17. The system of claim 16, wherein
one of the first therapeutic element and the second therapeutic element is positionable in the left brachiocephalic vein, and the other of the first therapeutic element and the second therapeutic element is positioned in the right brachiocephalic vein; and
the stimulator is configured to generate an electric field to modulate a cardiac nerve within a brachiocephalic triangle of a patient using anode and the cathode.

18. The system of claim 12, wherein the first therapeutic element and the second therapeutic element are each positionable in the left brachiocephalic vein.

19. The system of claim 12, wherein the first therapeutic element and the second therapeutic element are arranged on the at least one therapeutic element support to deliver the sympathetic therapy from a posterior portion of a distal part of the left brachiocephalic vein.

20. The system of claim 12, wherein the first therapeutic element and the second therapeutic element are each positionable in the right brachiocephalic vein.

21. The system of claim 16 further including a second anode positionable in a third blood vessel different from the first and second blood vessels, wherein the stimulator is further configured to generate a second electrode field to modulate an additional second cardiac nerve using the second anode and the cathode.
